(19) United States
(12) Patent Application Publication
(10) Pub. No.: US 2011/0295368 A1
(43) Pub. Date: Dec. 1, 2011

(54) ACCOMMODATIVE INTRAOCULAR LENS ASSEMBLY

(76) Inventor: Nir Betser, Yehud (IL)

(21) Appl. No.: 13/147,595

(22) PCT Filed: Jan. 31, 2010
(86) PCT No.: PCT/IB2010/050421

§ 371 Date: Aug. 3, 2011

Related U.S. Application Data
(60) Provisional application No. 61/150,762, filed on Feb. 8, 2009.

(51) Int. Cl. A61F 2/16

(52) U.S. Cl. 623/6.43

(57) ABSTRACT

An intraocular optic assembly including an anterior haptic member and a posterior haptic member arranged to correspond to anterior and posterior portions of a capsular bag, respectively, having an anterior-posterior axis passing centrally through the anterior and posterior haptic members, bendable link members attached to and between the anterior and posterior haptic members, an optic, and a lever pivotally connected at on end thereof to a first attachment point on a periphery of the optic and connected at an opposite end thereof to a second attachment point on one of the bendable link members, wherein linear movement of one of the anterior and posterior haptic members along the anterior-posterior axis applies a lever force on the optic with the lever that causes the optic to move linearly along the anterior-posterior axis.

### FIELD OF THE INVENTION

The present invention relates to intraocular lenses and particularly accommodative intraocular lenses.

### BACKGROUND OF THE INVENTION

Despite there being many accommodative intraocular lenses (AIOL) in the prior art, currently there is only one FDA approved AIOL and other AIOLs are in different phases of development. None of these AIOLs shows sufficient and continuous accommodation (change of optical power to focus on distance and near objects) with great optical quality.

### SUMMARY OF THE INVENTION

The present invention seeks to provide a unique AIOL that as a result of its exceptional design, can reach a level of accommodation that will enable patients to see up close, far away and everything in between without glasses.

The design restores the eye lens capsular bag and the natural accommodation mechanism in the eye after natural lens removal due to cataract or for refractive lens exchange (RLE).

The AIOL has a visual enhancement system that mimics, restores and exploits the natural accommodation mechanism in the eye.

An intraocular optic assembly including an anterior haptic member and a posterior haptic member arranged to correspond to anterior and posterior portions of a capsular bag, respectively, having an anterior-posterior axis passing centrally through the anterior and posterior haptic members, bendable link members attached to and between the anterior and posterior haptic members, an optic, and a lever pivotally connected at on end thereof to a first attachment point on a periphery of the optic and connected at an opposite end thereof to a second attachment point on one of the bendable link members, wherein linear movement of one of the anterior and posterior haptic members along the anterior-posterior axis applies a lever force on the optic with the lever that causes the optic to move linearly along the anterior-posterior axis.

The linear movement of one of the anterior and posterior haptic members causes the optic to move rotationally about the anterior-posterior axis.

The lever is arcuate and subtends an arc between the first and second attachment points of approximately 90°. The lever can be non-pivotally connected to the bendable link member. Each of the bendable link members includes a hinge, positioned between the anterior and posterior haptic members, about which the link member is bendable.

The anterior and posterior haptic members are sufficiently resilient to apply a force on a capsular bag that tends to restore a natural shape of the capsular bag.

A lock mechanism can be optionally provided that locks the anterior and posterior haptic members with respect to each other.

### BRIEF DESCRIPTION OF THE DRAWINGS

These and additional constructional features and advantages of the invention will be more readily understood in the light of the ensuing description of embodiments thereof, given by way of example only, with reference to the accompanying drawing wherein:

FIGS. 1A, 1B and 1C are simplified perspective, side, and top view illustrations, respectively, of an intraocular lens assembly, constructed and operative in accordance with an embodiment of the present invention;

FIGS. 2A, 2B and 2C are simplified perspective, side, and top view illustrations, respectively, of the IOL assembly of FIGS. 1A-1C, showing the optic moving in translatory motion between haptic members;

FIGS. 3A, 3B and 3C are simplified perspective, side, and top view illustrations, respectively, of the IOL assembly of FIGS. 1A-1C, showing the optic having reached the limit of its translatory travel;

FIGS. 4A, 4B, 4C, 4D, 4E and 4F are simplified left, side, front, right side, top, bottom perspective and top perspective view illustrations, respectively, of an intraocular lens assembly, constructed and operative in accordance with another embodiment of the present invention, having one lever and one bendable link member;

FIGS. 5A and 5B are simplified side view illustrations of the IOL assembly of FIGS. 4A-4F, showing the optic moving in translatory motion between haptic members;

FIG. 6 is a simplified perspective illustration of an intraocular lens assembly, constructed and operative in accordance with another embodiment of the present invention, with a haptic member comprising winged extensions;

FIG. 7 is a simplified perspective illustration of an intraocular lens assembly, constructed and operative in accordance with another embodiment of the present invention, with a haptic member comprising winged extensions and a lever pair; and

FIGS. 8A and 8B are simplified illustrations of reshaping of the capsular bag during accommodation and translatory motion of the optic, in accordance with an embodiment of the present invention.

### DETAILED DESCRIPTION OF EMBODIMENTS

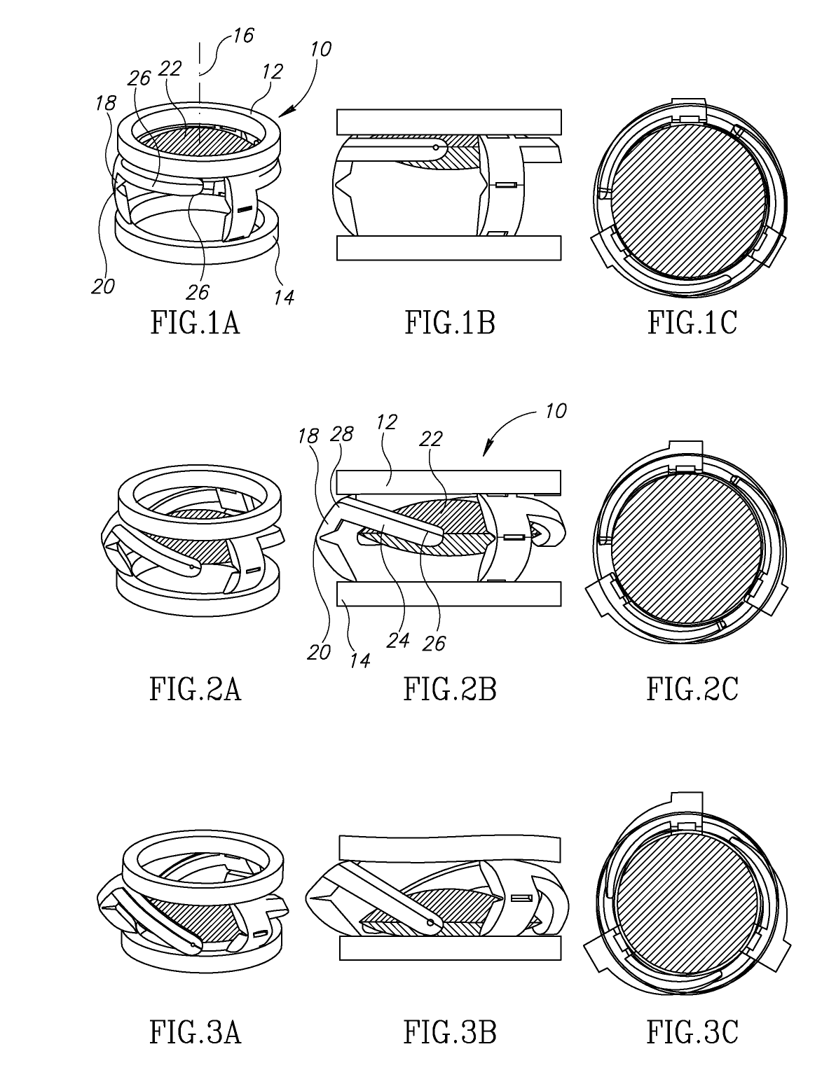

Reference is now made to FIGS. 1A-1C, which illustrate an IOL assembly 10, constructed and operative in accordance with a non-limiting embodiment of the present invention;

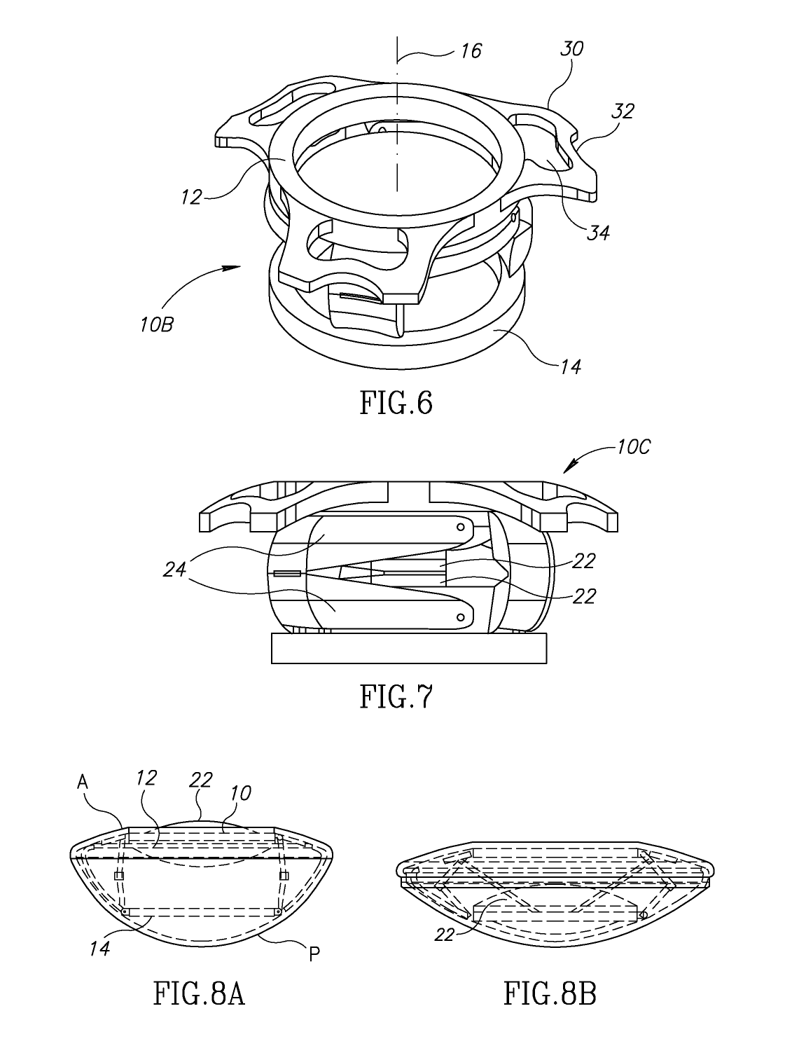

IOL assembly 10 includes an anterior haptic member 12 and a posterior haptic member 14 arranged to correspond to anterior and posterior portions A and P, respectively, of a capsular bag CB (not shown in FIGS. 1A-1C, but shown in FIG. 8A). Anterior and posterior haptic members 12 and 14 are shown as rings, but they can have other shapes as well, one of which is described further below with reference to FIG. 6. The rings are typical, but not necessarily, concentric. An anterior-posterior axis 16 (FIG. 1A) passes centrally through anterior and posterior haptic members 12 and 14.

Bendable link members 18 are attached to and between anterior and posterior haptic members 12 and 14. In the illustrated embodiment, each bendable link member 18 includes a hinge 20, positioned between anterior and posterior haptic members 12 and 14, about which link member 18 is bendable.

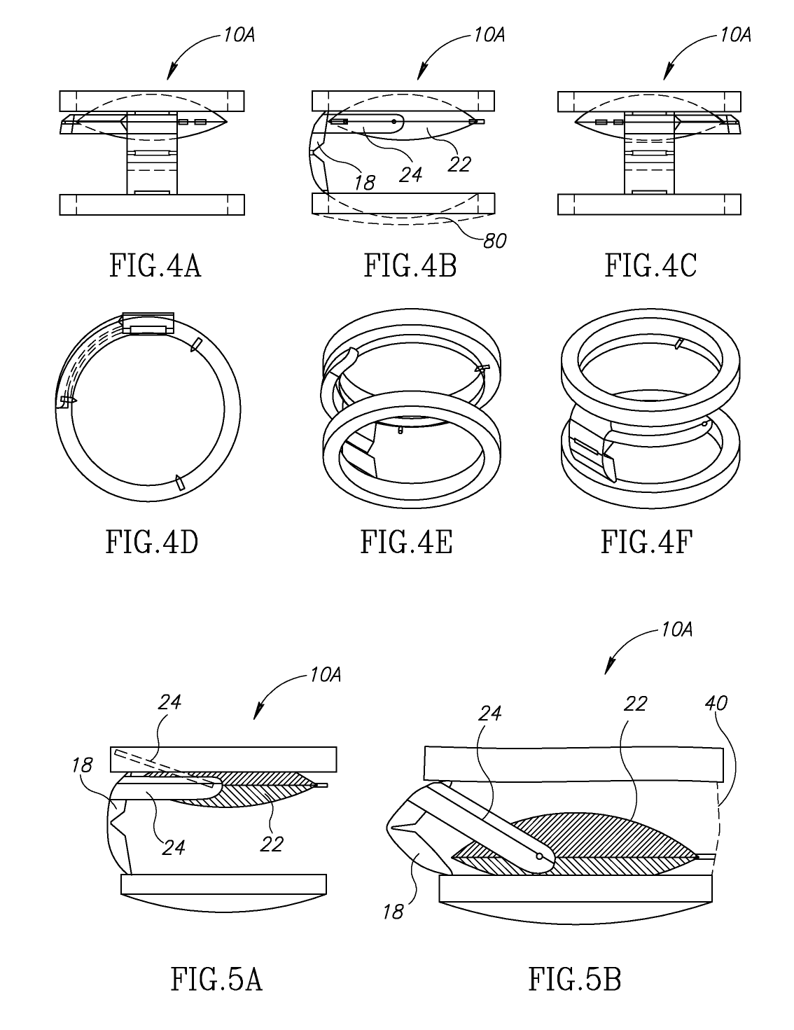

A lever 24 is pivotally connected at on end thereof to a first attachment point 26 on a periphery of an optic 22 and connected at an opposite end thereof to a second attachment point 28 (seen best in FIG. 2B) on one of the bendable link members 18. Lever 24 is preferably, but not necessarily, non-pivotally connected to link member 18. In the illustrated embodiment, there are three levers 24, spaced equally about axis 16. Each lever 24 is arcuate and subtends an arc L (FIG. 1C) between the first and second attachment points 26 and 28 of approximately 90° (±10° approximately). The invention is not limited to three levers 24, and may be carried out with any number of levers, even one (described further below with reference to FIGS. 4A-4F and 5A-5B).

When IOL assembly 10 is installed in an eye, accommodative structure of the eye (e.g., the ciliary processes and/or zonules) applies forces that cause movement of anterior and/or posterior haptic members 12/14. The linear movement of anterior and/or posterior haptic members 12/14 along anterior-posterior axis 16 applies a lever force on optic 22 with lever(s) 24, causing optic 22 to move linearly along anterior-posterior axis 16. The linear movement of the haptic members 12/14 generally causes optic 22 to move rotationally about anterior-posterior axis 16.

Although the invention is not limited to any theory of operation, in the present invention, although the forces of the eye structure, such as the ciliary processes and/or zonules, are located at the equator of the capsular bag, the accommodative forces cause the anterior and/or posterior portions of the bag to press on or otherwise act on the haptic members at the anterior and/or posterior portions of the bag (e.g., at the poles or near them). These accommodative forces move the haptic members to create the levered accommodated motion of the optic.

The movement of optic 22 can be easily seen by comparing its position in FIGS. 1A, 2A and 3A or FIGS. 1B, 2B and 3B.

Reference is now made to FIGS. 4A-4F, which illustrate an IOL assembly 10A, constructed and operative in accordance with another embodiment of the present invention. IOL assembly 10A differs from IOL assembly 10 in that IOL assembly 10A has only one lever 24 and one bendable link member 18. As seen in FIGS. 5A and 5B, in IOL assembly 10A, the optic 22 also moves in translatory motion between haptic members 12 and 14, in response to accommodative forces, as described before.

Optionally, for any of the embodiments of the invention, as shown in broken lines in FIG. 5A, lever 24 can be attached to either of the anterior and posterior haptic members 12 and 14. This may provide different accommodative movement of the optic 22.

Reference is now made to FIG. 6, which illustrates an IOL assembly 10B, constructed and operative in accordance with another embodiment of the present invention. IOL assembly 10B is basically the same as IOL assembly 10. In this embodiment, however, one of the haptic members, such as the anterior haptic member 12, includes winged extensions 30. In the illustrated embodiment, there are three winged extensions 30, spaced equally about axis 16. The invention is not limited to three winged extensions 30, and may be carried out with any number of winged extensions 30. Winged extensions 30, serving as extended haptics, stretch the capsular bag to its natural shape. The natural shape is generally considered important to ensure restoration of the natural accommodation mechanism in the eye after implantation. Winged extensions 30 also help to center the IOL in the capsular bag.

In the illustrated embodiment, each winged extension 30 is formed with a peripheral arcuate recess 32 and a cutout 34. Recess 32 helps the haptic sit firmly in the capsular bag and respond better to accommodative forces of the eye. Cutout 34 helps reduce weight and provide improved flexibility of the haptic. Moreover, each winged extension 30 is curved convexly, which makes it match better the curvature of the capsular bag.

Reference is now made to FIG. 7, which illustrates an IOL assembly 10C, constructed and operative in accordance with another embodiment of the present invention. IOL assembly 10C is basically the same as IOL assembly 10B. In this embodiment, however, there are a pair of levers 24 and a pair of optics 22. This may be used to provide greater optical power or telescopic effects (e.g., in the case of macular degeneration). The optics may be any combination of positive and/or negative lenses.

Reference is now made to FIGS. 8A and 8B, which illustrate reshaping of the capsular bag during accommodation and translatory motion of the optic 22, in accordance with an embodiment of the present invention. The anterior and posterior haptic members 12 and 14 are sufficiently resilient to apply a force on the capsular bag that tends to restore the natural shape of the capsular bag during the accommodative movement.

In summary, the IOL of the invention is structured to alter the distance between haptic members 12 and 14 under forces applied thereto from the capsular bag during natural accommodation. In the accommodated state (near distance vision) the IOL structure has a large separation between anterior and posterior haptic members 12 and 14 (creating large free space between them). In the non-accommodated state (far distance vision) the structure is narrow (slim) with a small separation between haptic members 12 and 14.

The IOL of the invention can use one or more (e.g., two) floating (cantilevered) lens complexes as the optic, thereby creating an optic structure with enhanced power, such as in the range between +15D and +25D (in steps of 0.25D or so), or more.

In accordance with another embodiment of the present invention, in addition to the floating optics, lenses (optics) 80 may be optionally placed at either or both of the anterior and posterior haptic members 12 and 14, as shown in broken lines in FIG. 4B. The additional lens or lenses 80 may form a doublet lens system with the floating optic.

The lens complexes can include fixed power optics, convex, concave, biconvex, biconcave, spherical and aspheric, astigmatic lenses, deformable optics, adjustable optics, aberration free optics, doublets, triplets, filtered optics, etc., and any combination thereof.

The shift of the floating lens during the accommodative process can be in the order of about 1 mm or more. The floating lens shift, posteriorly or anteriorly, results from the natural accommodation mechanism in the eye, and correctly focuses images on the retina.

Optionally, in order to control the capability of accommodation and to optimize the span of accommodation and/or to tense the zonules, the structure of the IOL assembly can be implanted in a pre-defined locked state with a lock mechanism 40 (shown in broken lines in FIG. 5B). For example, the IOL structure can be locked in a far distance vision state, or close to that state, but can be locked in any other pre-defined state. After implantation the lock mechanism 40 is disengaged and the IOL is ready to work as an accommodative IOL, exploiting the eye natural accommodation mechanism. The lock mechanism 40 can be designed to disengage a short time after implantation (from minutes to weeks or more). The lock mechanism 40 can include an absorbable suture, medical adhesive, a frangible structure or any other suitable means.

The IOL assembly can be fully pre-assembled or modularly assembled prior to or after implantation. The floating lens shape, size and mass can be designed to create minimum drag during movement while maintaining optimal optical performance.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. An intraocular lens assembly comprising:
an anterior haptic member and a posterior haptic member arranged to correspond to anterior and posterior portions of a capsular bag, respectively, having an anterior-posterior axis passing centrally through said anterior and posterior haptic members;
bendable link members attached to and between said anterior and posterior haptic members;
an optic; and
a lever pivotally connected at on end thereof to a first attachment point on a periphery of said optic and connected at an opposite end thereof to a second attachment point on one of said bendable link members, wherein linear movement of one of said anterior and posterior haptic members along the anterior-posterior axis applies a lever force on said optic with said lever that causes said optic to move linearly along the anterior-posterior axis.

2. The intraocular lens assembly according to claim 1, wherein said linear movement of one of said anterior and posterior haptic members causes said optic to move rotationally about the anterior-posterior axis.

3. The intraocular lens assembly according to claim 1, wherein said lever is arcuate and subtends an arc between said first and second attachment points of approximately 90°.

4. The intraocular lens assembly according to claim 1, wherein said lever is non-pivotally connected to said bendable link member.

5. The intraocular lens assembly according to claim 1, wherein each of said bendable link members comprises a hinge, positioned between said anterior and posterior haptic members, about which said link member is bendable.

6. The intraocular lens assembly according to claim 1, wherein said anterior and posterior haptic members are sufficiently resilient to apply a force on a capsular bag that tends to restore a natural shape of the capsular bag.

7. The intraocular lens assembly according to claim 1, further comprising a lock mechanism that locks said anterior and posterior haptic members with respect to each other.

8. The intraocular lens assembly according to claim 1, wherein at least one of said anterior and posterior haptic members comprises a winged extension.

9. The intraocular lens assembly according to claim 8, wherein said winged extension is formed with a peripheral arcuate recess and a cutout.

10. An intraocular lens assembly comprising:
an anterior haptic member and a posterior haptic member arranged to correspond to anterior and posterior portions of a capsular bag, respectively, having an anterior-posterior axis passing centrally through said anterior and posterior haptic members;
bendable link members attached to and between said anterior and posterior haptic members;
an optic; and
a lever pivotally connected at on end thereof to a first attachment point on a periphery of said optic and connected at an opposite end thereof to a second attachment point on one of said anterior and posterior haptic members, wherein linear movement of one of said anterior and posterior haptic members along the anterior-posterior axis applies a lever force on said optic with said lever that causes said optic to move linearly along the anterior-posterior axis.

11. The intraocular optic assembly according to claim 10, wherein said linear movement of one of said anterior and posterior haptic members causes said optic to move rotationally about the anterior-posterior axis.

12. The intraocular optic assembly according to claim 10, wherein said lever is arcuate and subtends an arc between said first and second attachment points of approximately 90°.

13. The intraocular optic assembly according to claim 10, wherein said lever is non-pivotally connected to said one of said anterior and posterior haptic members.

14. The intraocular optic assembly according to claim 10, wherein each of said bendable link members comprises a hinge, positioned between said anterior and posterior haptic members, about which said link member is bendable.

15. The intraocular optic assembly according to claim 10, wherein said anterior and posterior haptic members are sufficiently resilient to apply a force on a capsular bag that tends to restore a natural shape of the capsular bag.

16. The intraocular lens assembly according to claim 10, further comprising a lock mechanism that locks said anterior and posterior haptic members with respect to each other.

17. The intraocular lens assembly according to claim 10, wherein at least one of said anterior and posterior haptic members comprises a winged extension.

18. The intraocular lens assembly according to claim 17, wherein said winged extension is formed with a peripheral arcuate recess and a cutout.

19. The intraocular lens assembly according to claim 1, further comprising another optic placed at least one of the anterior and posterior haptic members.

20. The intraocular lens assembly according to claim 10, further comprising another optic placed at least one of the anterior and posterior haptic members.
